(19) United States
(12) Patent Application Publication
(10) Pub. No.: US 2021/0275354 A1
(43) Pub. Date: Sep. 9, 2021

(54) LACRIMAL INTUBATION SYSTEM

(71) Applicant: KANEKA CORPORATION, Osaka-shi, Osaka (JP)

(72) Inventor: Chihiro KOGA, Settsu-shi (JP)

(73) Assignee: KANEKA CORPORATION, Osaka-shi, Osaka (JP)

(21) Appl. No.: 17/261,276

(22) PCT Filed: Jul. 31, 2019
(86) PCT No.: PCT/JP2019/030008

§ 371 Date: Jan. 19, 2021

(30) Foreign Application Priority Data
Aug. 24, 2018 (JP) 2018-157488

(51) Int. Cl. A61F 9/007; A61B 1/07; A61M 27/00; A61B 1/32

(52) U.S. Cl. CPC A61F 9/00772; A61B 1/07; A61M 2210/0612; A61B 1/32; A61M 27/002

(57) ABSTRACT

A lacrimal intubation system including: a lacrimal duct tube having a first side and a second side, the first side and the second side being insertable into a lacrimal duct; an endoscope having an optical waveguide portion extending in a longitudinal direction, the optical waveguide portion being configured to be inserted into a lumen of the lacrimal duct tube; and a tubular member enclosing the optical waveguide portion.

### TECHNICAL FIELD

The present invention relates to a system that makes it easy to insert a lacrimal duct tube for relieving lacrimal duct obstruction into a lacrimal duct.

### BACKGROUND ART

Tears flow into the nasal cavity through the lacrimal duct which includes lacrimal puncta, lacrimal canaliculus, lacrimal sac, and nasolacrimal canal. When the lacrimal duct is obstructed, epiphora in which there is an overflow of tears may be caused. In order to relieve lacrimal duct obstruction, there is, for example, a method in which a lacrimal duct tube is placed in the lacrimal duct. Specifically, a method having two steps is commonly performed. In this method, an obstructed site is punctured using a lacrimal duct endoscope or a puncture bougie, and then, a lacrimal duct tube is placed in the obstructed site using an indwelling bougie. For example, Patent Document 1 discloses a lacrimal duct tube that is placed in the lacrimal duct using an insertion aid such as a bougie or a lacrimal duct endoscope. The tube has a distal end opening at the distal end, and a retaining portion for retaining the insertion aid is inserted into the tube in the vicinity of the opening. The retaining portion has a tubular structure in which both ends in the axial direction of the tube are open and which has an inner wall surface that guides and retains the insertion aid.

### RELATED ART DOCUMENTS

### Patent Documents

Patent Document 1: WO2013/111435

### SUMMARY OF THE INVENTION

### Technical Problem

However, when the indwelling bougie is removed from the lacrimal duct after the lacrimal duct tube as disclosed in Patent Document 1 is placed in the obstructed site, the indwelling bougie comes into contact with the inner wall of the lacrimal duct tube, so that the lacrimal duct tube is pulled toward the operator's hand. Thus, the position of the lacrimal duct tube may be displaced from the obstructed site. In addition, in a case where the insertion aid is a bougie, there is also a problem that the state of the obstructed site cannot be observed when the lacrimal duct tube is placed. In view of this, an object of the present invention is to provide a lacrimal intubation system that prevents positional displacement of a lacrimal duct tube when the insertion aid is removed, and that enables observation of the state inside the lacrimal duct.

### Solutions to the Problems

The gist of a lacrimal intubation system according to the present invention that can overcome the above problems is as follows. A lacrimal intubation system includes a lacrimal duct tube having a first side and a second side, the first side and the second side being insertable into a lacrimal duct; an endoscope having an optical waveguide portion extending in a longitudinal direction, the optical waveguide portion being configured to be inserted into a lumen of the lacrimal duct tube; and a tubular member enclosing the optical waveguide portion.

In the lacrimal intubation system, the tubular member is preferably disposed in the lumen of the lacrimal duct tube.

In the lacrimal intubation system, the optical waveguide portion preferably has a distal end located distal to a distal end of the tubular member.

In the lacrimal intubation system, the lacrimal duct tube is preferably configured to move distally or proximally to the endoscope, and the tubular member is preferably configured not to move distally or proximally to the endoscope.

In the lacrimal intubation system, the tubular member preferably has a distal end, and at the distal end, a gap between the tubular member and the optical waveguide portion is preferably 0.5 times or more and 2 times or less a gap between the tubular member and the lacrimal duct tube in a radial direction of the tubular member.

In the lacrimal intubation system, the optical waveguide portion preferably has a distal end located distal to a first end or a second end of the lacrimal duct tube.

In the lacrimal intubation system, the tubular member preferably has a proximal end aligned with a proximal end of the optical waveguide portion.

In the lacrimal intubation system, the tubular member preferably has a proximal end located distal to a proximal end of the optical waveguide portion.

In the lacrimal intubation system, at least a part of the optical waveguide portion is preferably made of metal.

In the lacrimal intubation system, the lacrimal duct tube preferably has a first tubular portion located at the first side, a second tubular portion located at the second side, and a central portion located between the first tubular portion and the second tubular portion, and each of the first tubular portion and the second tubular portion preferably has a scale for specifying the position of the lacrimal duct tube in the longitudinal direction.

In the lacrimal intubation system, the tubular member is preferably made of at least one of a polyimide resin, a fluorine resin, a polyethylene resin, and a polyamide resin.

In the lacrimal intubation system, the tubular member preferably has a slit, and is preferably made of metal.

### Advantageous Effects of the Invention

According to the present invention, since the optical waveguide portion is covered with the tubular member, slipperiness of the endoscope with respect to the lacrimal duct tube is enhanced. Therefore, even if the optical waveguide portion and the inner wall of the lacrimal duct tube come into contact with each other, the endoscope is easily removed from the lacrimal duct tube. Therefore, it is possible to prevent the lacrimal duct tube from being pulled toward the operator's hand together with the endoscope and being displaced from the obstructed site. In addition, since the lacrimal duct tube can be placed using the endoscope, it becomes easier to observe the inside of the lacrimal duct during placement.

### BRIEF DESCRIPTION OF DRAWINGS

FIG. 1 is a schematic view showing an inside of a lacrimal duct.

FIG. 2 is a side view (partially in cross section) of the lacrimal intubation system according to an embodiment of the present invention.

FIG. 3A is an enlarged side view (partially in cross section) of a distal portion of the lacrimal intubation system according to an embodiment of the present invention.

FIG. 3B is an enlarged side view (partially in cross section) of a distal portion of the lacrimal intubation system according to an embodiment of the present invention.

FIG. 3C is an enlarged side view (partially in cross section) of a distal portion of the lacrimal intubation system according to an embodiment of the present invention.

FIG. 3D is an enlarged side view (partially in cross section) of a distal portion of the lacrimal intubation system according to an embodiment of the present invention.

FIG. 4 is a side view (partially in cross section) showing a modification of the lacrimal intubation system according to an embodiment of the present invention.

FIG. 5A is a side view (partially in cross section) showing a modification of the lacrimal intubation system according to an embodiment of the present invention.

FIG. 5B is a side view (partially in cross section) showing a modification of the lacrimal intubation system according to an embodiment of the present invention.

FIG. 5C is a side view (partially in cross section) showing a modification of the lacrimal intubation system according to an embodiment of the present invention.

FIG. 6 is a sectional view of a lacrimal duct tube according to an embodiment of the present invention.

FIG. 7 is a sectional view showing a modification of a lacrimal duct tube according to an embodiment of the present invention.

FIG. 8 is a front view of an endoscope according to an embodiment of the present invention as viewed from a distal end.

FIG. 9 is a sectional view of a tubular member according to an embodiment of the present invention

FIG. 10 is a side view showing a modification of the tubular member according to an embodiment of the present invention.

FIG. 11A is a plan view showing another example of the lacrimal duct tube according to an embodiment of the present invention.

FIG. 11B is a side view showing another example of the lacrimal duct tube according to an embodiment of the present invention.

### DESCRIPTION OF EMBODIMENTS

The present invention will be specifically explained below based on the following embodiments, however, the present invention is not restricted by the embodiments described below of course, and can be certainly put into practice after appropriate modifications within in a range meeting the gist of the above and the below, all of which are included in the technical scope of the present invention. In the drawings, hatching, a reference sign for a member may be omitted for convenience, and in such a case, the description and other drawings should be referred to. In addition, sizes of various members in the drawings may differ from the actual sizes thereof, since priority is given to understanding the features of the present invention.

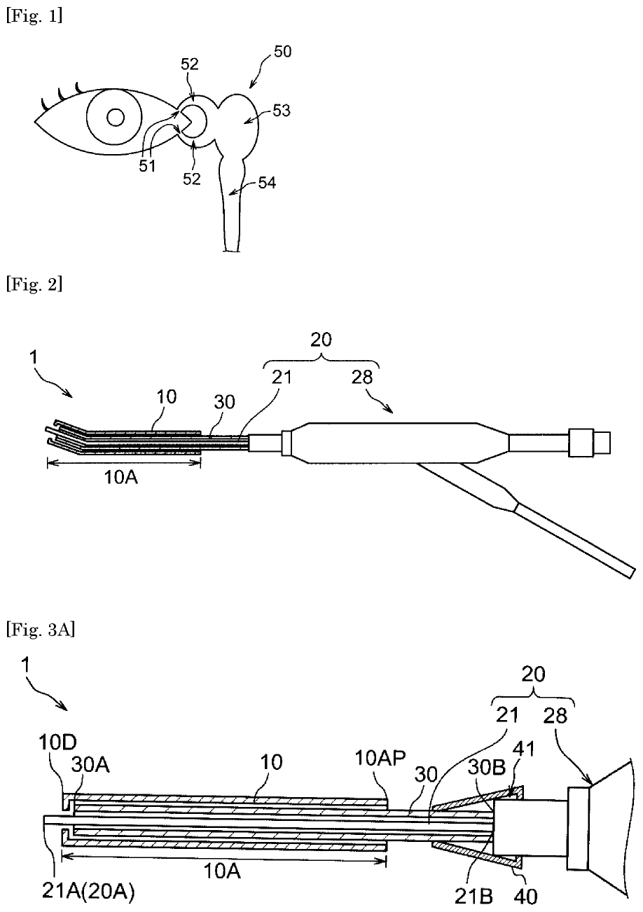

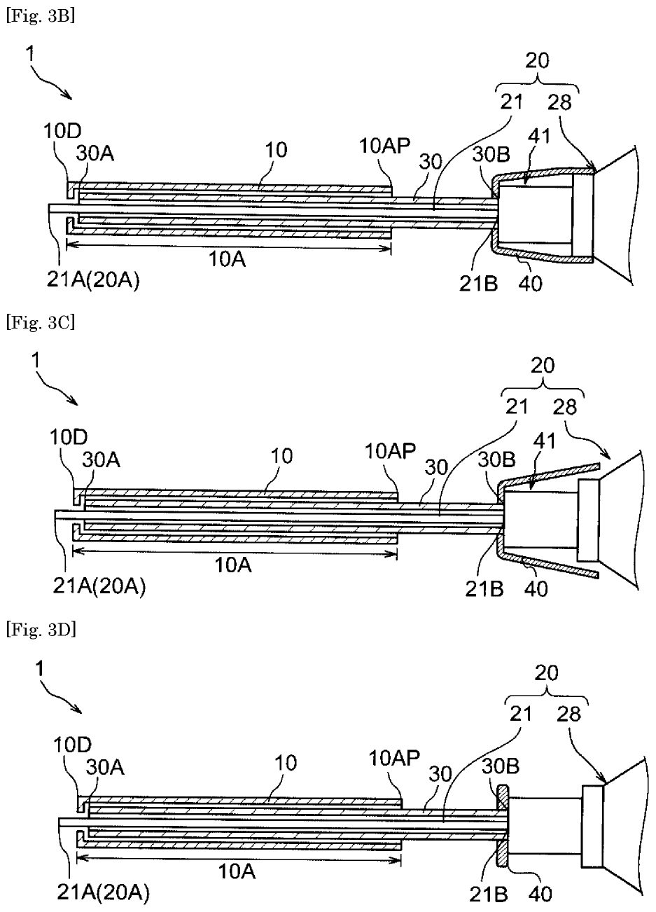

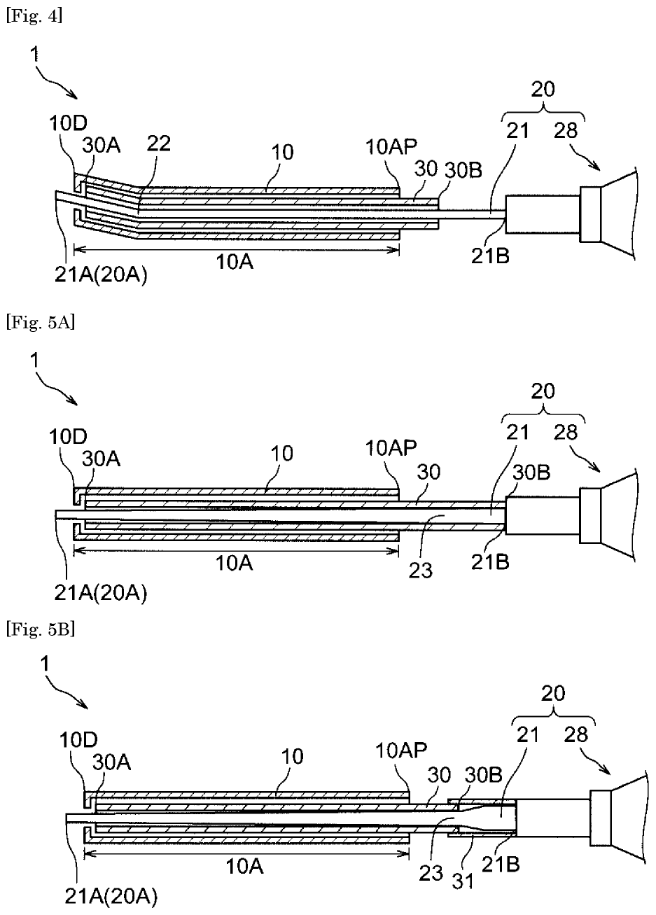

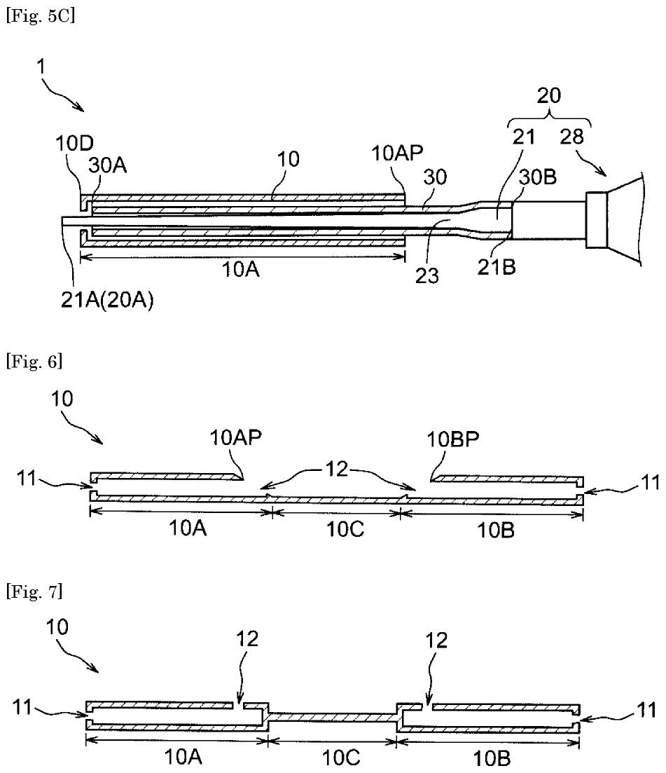

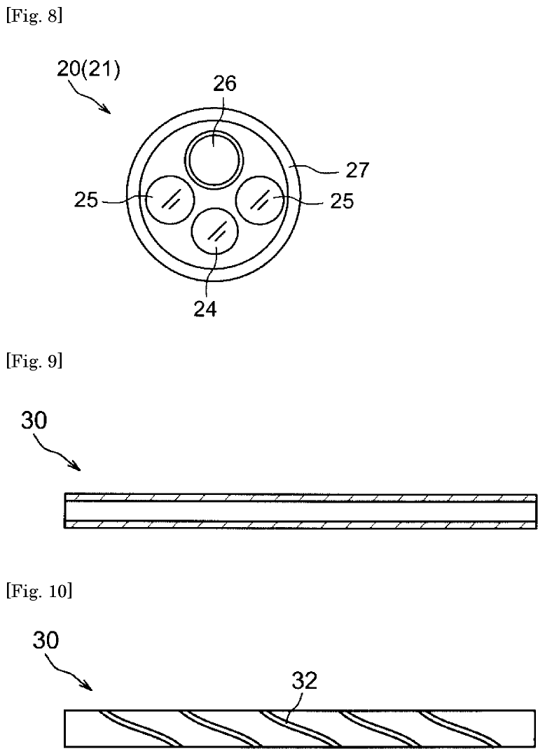

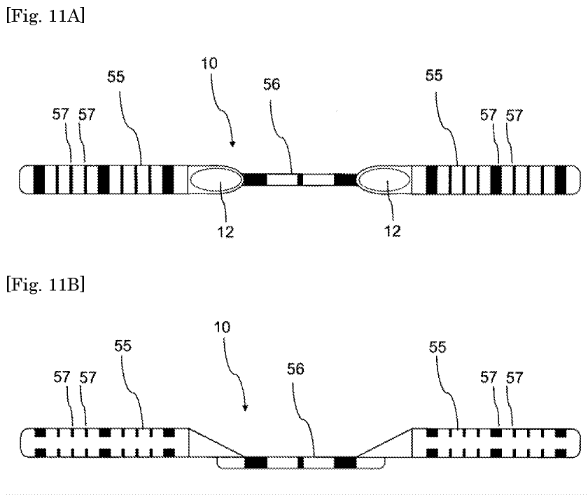

The configuration of a lacrimal intubation system will be described with reference to FIGS. 1 to 11. FIG. 1 is a schematic view showing an inside of a lacrimal duct. FIG. 2 is a side view (partially in cross section) of the lacrimal intubation system, and FIGS. 3A to 3D are enlarged side views (partially in cross section) of a distal portion of the lacrimal intubation system shown in FIG. 2. FIGS. 4 and 5 are side views (partially in cross section) showing modifications of the lacrimal intubation system shown in FIGS. 3A to 3D. FIGS. 6 and 7 are sectional views of a lacrimal duct tube according to the embodiment of the present invention. FIG. 8 is a front view of an endoscope according to the embodiment of the present invention as viewed from a distal end. FIG. 9 is a sectional view of a tubular member according to the embodiment of the present invention, and FIG. 10 is a side view showing a modification of the tubular member shown in FIG. 9. FIGS. 11A and 11B show modes in which the lacrimal duct tube according to the embodiment of the present invention is marked with a scale. As shown in FIGS. 2 and 3, a lacrimal intubation system 1 includes a lacrimal duct tube 10, an endoscope 20, and a tubular member 30. Further, as shown in FIG. 2, an optical waveguide portion 21 of the endoscope 20 used for examining the inside of the lacrimal duct usually has a portion that bends at a predetermined angle at a distal part. It is to be noted that, since the system according to the embodiment of the present invention does not depend on the shape of the optical waveguide portion 21, some of the optical waveguide portions 21 shown in FIGS. 3 to 10 have a linear shape, and some have a bent shape. However, the present invention can be embodied no matter what shape the optical waveguide portion 21 has. FIGS. 2 to 5 show a state in which the optical waveguide portion 21 of the endoscope 20 is inserted into the lumen of the lacrimal duct tube 10 shown in FIG. 6. In the following, the lacrimal intubation system may be referred to as a “system” and the lacrimal duct tube may be referred to as a “tube”.

The lacrimal duct tube 10 is intubated into a lacrimal duct 50 to maintain a route within the lacrimal duct 50. The lacrimal duct tube 10 has a first side and a second side, and the first side and the second side are both intubated into the lacrimal duct 50. In the following, the direction from the first side to the second side of the lacrimal duct tube 10 is referred to as an extension direction of the tube 10.

The lacrimal duct tube 10 may be at least partially tubular in the extension direction, and may be at least partially solid in the extension direction. As shown in FIGS. 6 and 7, the lacrimal duct tube 10 includes a first section 10A including a first end part, a second section 10B including a second end part, and a central section 10C located between the first section 10A and the second section 10B in the extension direction. In that case, the lacrimal duct tube 10 may be tubular in the first section 10A and the second section 10B, and may be solid in the central section 10C. In the extension direction of the tube 10, the first section 10A and the second section 10B may be shorter than the central section 10C.

The lacrimal duct tube 10 is preferably flexible so that it can be inserted and placed in the lacrimal duct 50, and can be made of a synthetic resin such as a polyurethane resin, a polyethylene resin, a polyamide resin, a silicone resin, a fluorine resin such as polytetrafluoroethylene, or an isobutylene copolymer resin, or a combination thereof.

The lacrimal duct tube 10 may have a single layer or a plurality of layers. A part of the tube 10 in the extension direction may have a single layer, and another part may have a plurality of layers. For example, in a case where the lacrimal duct tube 10 has the first section 10A, the second section 10B, and the central section 10C, the first section 10A and the second section 10B may have a plurality of layers, and the central section 10C may have a single layer. Further, the lacrimal duct tube 10 may have a single layer on the first end part and the second end part, and may have a plurality of layers in the section excluding the first end part and the second end part. When the lacrimal duct tube 10 is at least partially composed of a plurality of layers as described above, the hardness of the tube 10 and the slipperiness of the tube 10 with respect to another member can be increased.

As shown in FIGS. 6 and 7, it is preferable that the lacrimal duct tube 10 is provided with an insertion port 12 for inserting the endoscope 20 in the middle of the tube 10 in the extension direction. It is more preferable that the insertion ports 12 are provided in the first section 10A and the second section 10B, and it is further preferable that the insertion ports 12 are provided on the end of the first section 10A close to the central section 10C and the end of the second section 10B close to the central section 10C. As shown in FIG. 6, the insertion ports 12 may be provided at an end surface (proximal end surface 10AP) of the first section 10A on the central section 10C side and an end surface (proximal end surface 10BP) of the second section 10B on the central section 10C side. Further, as shown in FIG. 7, the insertion ports 12 may be provided at a position distal to the proximal end surface 10AP of the first section 10A and at a position distal to the proximal end surface 10BP of the second section 10B. The insertion port 12 can be formed by providing a notch or an opening in the lacrimal duct tube 10. The shape of the insertion port 12 is not particularly limited, but it is preferable that the longitudinal direction of the insertion port 12 extends along the extension direction of the tube 10.

As shown in FIGS. 6 and 7, it is preferable that an opening 11 is provided in at least one of the first end part and the second end part of the lacrimal duct tube 10. Since the outside of the lacrimal duct tube 10 and the lumen of the lacrimal duct tube 10 communicate with each other through the opening 11, it becomes easy to observe the inside of the lacrimal duct 50 when the endoscope 20 is inserted into the lacrimal duct tube 10. In addition, tears pass through the opening 11 without accumulating after the lacrimal duct tube 10 is placed, so that cleanliness can be maintained. In addition, since the flow path is increased, it is expected that lacrimation will be further improved. It is more preferable that the openings 11 are provided at the first end part and the second end part of the lacrimal duct tube 10, and it is further preferable that the lacrimal duct tube 10 has the opening 11 when viewed from the first end side or the second end side.

The diameter of the opening 11 is preferably smaller than the outer diameter of the lacrimal duct tube 10 at the first end or the second end. With this configuration, a distal end 30A of the tubular member 30 is easy to abut the first end part or the second end part of the lacrimal duct tube 10, whereby excessive protrusion of the endoscope 20 from the opening 11 of the lacrimal duct tube 10 can be prevented.

The shape of the opening 11 is not particularly limited, and the opening 11 may have a circular shape, an elliptical shape, a polygonal shape, or a shape obtained by combining these shapes.

As shown in FIGS. 6 and 7, the outer diameter of the lacrimal duct tube 10 in the first section 10A and the second section 10B may be larger than the outer diameter in the central section 10C. Due to the configuration described above, the tube 10 has a so-called Nunchaku style. Therefore, it is easy to locate the first section 10A and the second section 10B between a lacrimal sac 53 and a nasolacrimal canal 54, and to locate the central section 10C between a lacrimal punctum 51 and a lacrimal canaliculus 52, whereby the tube 10 can be stably placed in the lacrimal duct 50.

As shown in FIG. 2, in order to insert the optical waveguide portion 21 of the endoscope 20 into the lumen of the lacrimal duct tube 10, the inner diameter of the lacrimal duct tube 10 in the first section 10A and the second section 10B can be set greater than the outer diameter of the optical waveguide portion 21 of the endoscope 20.

The length and outer diameter of the lacrimal duct tube 10 can be set according to the size of the lacrimal duct 50. For example, the length of the lacrimal duct tube 10 can be set to 5 cm or more and 15 cm or less, the outer diameter can be set to 0.5 mm or more and 1.7 mm or less, and the inner diameter can be set to 0.5 mm or more and 0.9 mm or less.

The lacrimal duct tube 10 is preferably made of a transparent or semi-transparent material in order that the position of the endoscope 20 within the lacrimal duct tube 10 is easily recognized. Further, in order to easily ensure the field of view by the endoscope 20, it is preferable that the first end surface and the second end surface of the lacrimal duct tube 10 are made of a transparent material.

The lacrimal duct tube 10 preferably includes a material having a shore A hardness of 60 or more, more preferably 70 or more, still more preferably 80 or more, and most preferably 85 or more. Further, the lacrimal duct tube 10 may include a material having a shore A hardness of 100 or less. When the lacrimal duct tube 10 includes such a material, appropriate hardness can be applied to the lacrimal duct tube 10, which facilitates insertion of the tube 10 into the lacrimal duct 50. Note that the shore A hardness is measured according to the ISO868: 2003 plastic durometer hardness test method.

It is preferable that the shore A hardness of the lacrimal duct tube 10 at the first end part and the second end part is higher than the shore A hardness in the section excluding the first end part and the second end part. Due to the lacrimal duct tube 10 having appropriate rigidity at the first end part and the second end part, excessive protrusion of the endoscope 20 from the opening 11 of the lacrimal duct tube 10 can be prevented. In order to facilitate the placement of the tube 10 in the lacrimal duct 50, it is preferable that the shore A hardness in the central section 10C is lower than the shore A hardness in the first section 10A and the second section 10B. The shore A hardness in the present specification means an average value of the shore A hardness measured from a first side surface side of the lacrimal duct tube 10 and the shore A hardness measured from a second side surface side which is the back side of the first side surface.

In a case where the lacrimal duct tube 10 has the first section 10A, the second section 10B, and the central section 10C, materials constituting the respective sections may be the same or different.

The thickness of the lacrimal duct tube 10, especially in the first section 10A and the second section 10B, is preferably 40 μm or more, more preferably 50 μm or more, and still more preferably 60 m or more. Further, it is preferably 150 μm or less, more preferably 120 μm or less, and still more preferably 100 μm or less. This configuration can prevent the lacrimal duct tube 10 from being ruptured while maintaining the flexibility of the lacrimal duct tube 10.

The endoscope 20 has the optical waveguide portion 21 extending in the longitudinal direction, and the optical waveguide portion 21 is inserted into the lumen of the lacrimal duct tube 10. It is preferable that at least a part (preferably, the distal portion) of the optical waveguide portion 21 is inserted into the lacrimal duct 50, and an optical fiber is inserted into at least a part of the optical waveguide portion 21 in the longitudinal direction. Here, the proximal portion of the endoscope 20 indicates a side closer to the user, that is, the operator's hand, with respect to the extension direction of the optical waveguide portion 21, and the distal portion indicates the side opposite to the proximal portion, that is, the side closer to a treatment target. Further, a direction from the proximal portion to the distal portion of the endoscope 20 is referred to as the longitudinal direction.

In FIG. 2, the endoscope 20 includes the optical waveguide portion 21 which is at least partially inserted into the lacrimal duct 50, and a proximal-side operating portion 28 disposed proximal to the optical waveguide portion 21. In FIG. 8, the optical waveguide portion 21 of the endoscope 20 has an outer tube 27, and various members constituting the endoscope 20 are disposed in the outer tube 27. The outer tube 27 may include an objective optical system including an imaging element, an objective lens 24 located distal to the imaging element, and a control circuit connected to the imaging element, and an illumination optical system including an illumination lens 25, an optical fiber connected to the illumination lens 25, and a condenser lens disposed proximal to the optical fiber, for example. Further, the outer tube 27 may include inside a liquid injection channel 26 for supplying physiological saline or the like into the lacrimal duct 50.

The imaging device of the endoscope 20 is not particularly limited, and the endoscope 20 may be a fiberscope or an electronic scope. For example, in the objective optical system, an optical fiber may be used instead of the imaging element. The optical fiber used in the objective optical system or the illumination optical system can be made of glass, plastic, or the like.

The endoscope 20 is not particularly limited as long as it can be inserted into the lacrimal duct 50, and a commercially available lacrimal endoscope such as a lacrimal duct fiberscope manufactured by FiberTech Co., Ltd. can be used.

The length of the optical waveguide portion 21 of the endoscope 20 in the longitudinal direction can be set according to the length of the lacrimal duct 50 of a patient, and can be set to, for example, 40 mm or more, 50 mm or more, or 60 mm or more, or 100 mm or less, 90 mm or less, or 80 mm or less.

The outer diameter of a distal end 21A of the optical waveguide portion 21 of the endoscope 20 can be set to, for example, 0.5 mm or more, 0.6 mm or more, or 0.7 mm or more, or 1.2 mm or less, 1.1 mm or less, or 1.0 mm or less. Further, the maximum outer diameter of the optical waveguide portion 21 of the endoscope 20 can be set to, for example, 0.8 mm or more, 0.9 mm or more, or 1.0 mm or more, or 1.5 mm or less, 1.4 mm or less, or 1.3 mm or less.

At least a part of the optical waveguide portion 21 is preferably made of metal, and can be made of, for example, stainless steel, Ni—Ti alloy, or the like. Specifically, it is preferable that the outer tube 27 constituting the optical waveguide portion 21 is made of metal. By using the outer tube 27 made of metal as described above, appropriate rigidity can be applied to the optical waveguide portion 21, so that the optical waveguide portion 21 can easily puncture and penetrate the obstructed site.

A modification of the optical waveguide portion 21 of the endoscope 20 will be described with reference to FIG. 4. As shown in FIG. 4, a bending portion 22 that bends outward in the diametrical direction of the optical waveguide portion 21 may be provided at the distal end part of the optical waveguide portion 21 of the endoscope 20. With this configuration, the optical waveguide portion 21 can easily follow the shape of the lacrimal duct 50, whereby the optical waveguide portion 21 can be easily inserted into and removed from the lacrimal duct 50.

As shown in FIG. 5A, it is preferable that a large-diameter portion 23 in which the outer diameter of the optical waveguide portion 21 of the endoscope 20 is equal to or greater than the inner diameter of the tubular member 30 is formed on a proximal portion (more preferably, formed distal to the proximal end) of the optical waveguide portion 21. With this configuration, when the optical waveguide portion 21 is inserted into the lumen of the tubular member 30, the large-diameter portion 23 abuts against the inner wall of the tubular member 30 to fix the endoscope 20 and the tubular member 30. Further, when the outer diameter of the optical waveguide portion 21 is increased on the proximal portion as in the large-diameter portion 23, it is possible to prevent the optical waveguide portion 21 from breaking at this portion.

Examples of a structure for forming the large-diameter portion 23 on the optical waveguide portion 21 include a mode in which the outer diameter of the optical waveguide portion 21 is increased toward the proximal portion, and a mode in which a flange having an outer diameter greater than that of the distal end of the optical waveguide portion 21 is provided on the proximal portion of the optical waveguide portion 21.

In addition, the tubular member 30 and a distal end part of the operating portion 28 can be fixed using a tubular member 31 having an outer diameter greater than that of the tubular member 30 as shown in FIG. 5B, or the diameter of the proximal portion of the tubular member 30 can be increased by flaring to bring the proximal end 30B of the tubular member 30 into contact with the distal end of the operating portion 28 as shown in Fig. 5C.

The above embodiment describes an example in which the endoscope 20 is used as an intubation aid of the lacrimal duct tube 10. However, instead of the endoscope 20, another equipment for intubation such as a bougie (also referred to as a probe), a stylet, a laser irradiation device, a catheter, or a catheter with balloon can also be used.

The bougie or stylet preferably has an insertion portion formed in a rod shape and an operating portion that is disposed proximal to the insertion portion and is gripped by the operator. In that case, it is preferable that the tubular member 30 is provided so as to enclose the insertion portion of the bougie or stylet. The insertion portion is preferably made of a metal material. The distal end of the insertion portion is preferably curved in order to reduce a stimulus when the distal end of the insertion portion contacts the inner wall of the lacrimal duct 50.

Similar to the endoscope 20, the laser irradiation device preferably has an insertion portion to be inserted into the lacrimal duct 50 and an operating portion that is disposed proximal to the insertion portion and is gripped by the operator. In that case, it is preferable that the tubular member 30 is provided so as to enclose the insertion portion of the laser irradiation device.

The catheter preferably has a tubular shaft portion extending in the longitudinal direction and an operating portion that is disposed proximal to the shaft portion and is gripped by the operator. In that case, it is preferable that the tubular member 30 is provided so as to enclose the shaft portion of the catheter.

The catheter may have a balloon. That is, the catheter may have a tubular shaft portion extending in the longitudinal direction, a balloon provided on a distal portion of the shaft portion and outside of the shaft portion in the radial direction, and an operating portion connected to the proximal portion of the shaft. In that case, it is preferable that the tubular member 30 is provided proximal to the proximal end of the balloon, and the portion where the tubular member 30 is provided is located in the lumen of the lacrimal duct tube 10. Accordingly, the operator can perform a procedure of inflating the balloon to eliminate the obstruction in the lacrimal duct 50, and then, deflating the balloon, and moving the catheter further distally to deliver the lacrimal duct tube 10 to a desired position.

The tubular member 30 is a member provided to improve the slipperiness of the endoscope 20 with respect to the lacrimal duct tube 10, and encloses the optical waveguide portion 21 of the endoscope 20 as shown in FIGS. 2 and 3. Since the optical waveguide portion 21 is covered with the tubular member 30, the slipperiness of the endoscope 20 with respect to the lacrimal duct tube 10 is enhanced. Therefore, even if the optical waveguide portion 21 and the inner wall of the lacrimal duct tube 10 come into contact with each other, the endoscope 20 is easily removed from the lacrimal duct tube 10. Therefore, it is possible to prevent the lacrimal duct tube 10 from being pulled toward the operator's hand together with the endoscope 20 and being displaced from the obstructed site. Since the lacrimal duct tube 10 can be placed using the endoscope 20, it becomes easier to observe the inside of the lacrimal duct 50 during placement.

The tubular member 30 is preferably located in the lumen of the lacrimal duct tube 10. This configuration provides satisfactory slipperiness of the optical waveguide portion 21 of the endoscope 20 with respect to the inner wall of the lacrimal duct tube 10. It is more preferable that the tubular member 30 is located in the lumen of the lacrimal duct tube 10 in the first section 10A or the second section 10B. Note that FIGS. 2 and 3 show an example in which the optical waveguide portion 21 of the endoscope 20 and the tubular member 30 are located in the lumen of the lacrimal duct tube 10 in the first section 10A.

It is preferable that at least a part of the optical waveguide portion 21 of the endoscope 20 in the longitudinal direction is covered with the tubular member 30, and it is more preferable that the portion of the endoscope 20 proximal to the distal end of the endoscope 20 is covered with the tubular member 30. That is, it is preferable that, as shown in FIGS. 3A to 3D, a distal end 20A of the endoscope 20 is disposed distal to the distal end 30A of the tubular member 30. This configuration can prevent a situation in which image capture is difficult because of foreign matters such as a tear stone entering a gap between the endoscope 20 and the tubular member 30 or a film-shaped obstructed site adhering to the distal end part of the tubular member 30. Further, when the endoscope 20 is removed from the lacrimal duct 50, damage condition of the distal end part of the endoscope 20 can be easily recognized without removing the tubular member 30 from the endoscope 20.

It is preferable that, as shown in FIGS. 3A to 3D, the tubular member 30 encloses the optical waveguide portion 21 from a position proximal to the distal end 20A (distal end 21A of the optical waveguide portion 21) of the endoscope 20 to a proximal end 21B of the optical waveguide portion 21. Specifically, it is preferable that the proximal end 30B of the tubular member 30 is aligned with the proximal end 21B of the optical waveguide portion 21. With this configuration, the slipperiness of the endoscope 20 with respect to the lacrimal duct tube 10 is improved in the entire portion of the optical waveguide portion 21 of the endoscope 20 except for the distal end 21A, whereby the displacement of the lacrimal duct tube 10 is further suppressed.

It is only sufficient that, as shown in FIGS. 4 and 5, the tubular member 30 encloses the optical waveguide portion 21 from the position proximal to the distal end 20A (distal end 21A of the optical waveguide portion 21) of the endoscope 20 to a position distal to the proximal end 21B of the optical waveguide portion 21. Specifically, it is only sufficient that the proximal end 30B of the tubular member 30 is located distal to the proximal end 21B of the optical waveguide portion 21. In that case, it is preferable that the length of the tubular member 30 in the longitudinal direction is greater than the length of the section of the lacrimal duct tube 10 inserted into the optical waveguide portion 21.

It is preferable that the distal end 20A of the endoscope 20 is located distal to the first end or the second end of the lacrimal duct tube 10. Specifically, it is preferable that the distal end 21A of the optical waveguide portion 21 is located distal to the first end or the second end of the lacrimal duct tube 10. With this configuration, when the system 1 is inserted into the lacrimal duct 50, the endoscope 20 precedes, so that the lacrimal duct tube 10 can be placed while penetrating the obstructed site in the lacrimal duct 50. Note that FIGS. 3A to 3D show examples in which the distal end 20A of the endoscope 20 is located distal to the first end 10D of the lacrimal duct tube 10.

It is preferable that the distal end 21A of the optical waveguide portion 21 is disposed distal to the distal end 30A of the tubular member 30. With this configuration, when the system 1 is inserted into the lacrimal duct 50, the optical waveguide portion 21 precedes, so that the lacrimal duct tube 10 can be placed while penetrating the obstructed site in the lacrimal duct 50.

It is preferable that the distal end 20A of the endoscope 20 (more preferably, the distal end 21A of the optical waveguide portion 21) is located distal to the distal end 30A of the tubular member 30 and distal to the first end or the second end of the lacrimal duct tube 10. With this configuration, when the system 1 is inserted into the lacrimal duct 50, the endoscope 20 precedes, so that the lacrimal duct tube 10 can be placed while penetrating the obstructed site in the lacrimal duct 50.

It is preferable that the lacrimal duct tube 10 move distally or proximally to the endoscope 20, and the tubular member 30 does not move distally or proximally to the endoscope 20. When the endoscope 20 is moved proximally, the tubular member 30 also moves proximally together with the endoscope 20. Thus, this configuration can prevent the tubular member 30 from remaining in the lacrimal duct 50.

In order to prevent the tubular member 30 from moving distally or proximally to the endoscope 20, it is preferable that the endoscope 20 and the tubular member 30 are fixed. The position where the endoscope 20 and the tubular member 30 are fixed is not particularly limited, but it is preferable that the tubular member 30 is fixed to a position distal to the proximal end of the optical waveguide portion 21. With this configuration, even when the distal portion of the optical waveguide portion 21 is inserted into the lacrimal duct 50, the fixing state between the endoscope 20 and the tubular member 30 is easily checked.

The method for fixing the endoscope 20 and the tubular member 30 is not particularly limited. For example, as shown in FIG. 3A, a fixing member 40 that encloses the proximal end part of the optical waveguide portion 21 of the endoscope 20, the distal end part of the operating portion 28, and the proximal end part of the tubular member 30 may be provided. When the fixing member 40 is provided, the tubular member 30 is pressed inward in the radial direction of the optical waveguide portion 21, so that the tubular member 30 is less likely to move distally or proximally. The fixing member 40 is preferably a protective cap having a hollow portion 41. The fixing member 40 can be made of an elastic material such as silicone rubber or a polyamide resin. As the fixing member 40, various modes shown in FIGS. 3B to 3D can be employed. For example, when the fixing member 40 is fixed to both the tubular member 30 and the endoscope 20 as shown in FIG. 3B, the operator can perform the procedure by gripping the operating portion 28. The operator can also perform the procedure even when the fixing member 40 is fixed to the tubular member 30 without being fixed to the endoscope 20 as shown in FIG. 3C. In this case, the operator preferably performs the procedure by gripping the operating portion 28 while holding a free end of the fixing member 40 with fingers or the like. Further, the operator can also perform the procedure even when the fixing member 40 is fixed to the tubular member 30 and the distal part of the operating portion 28 as shown in FIG. 3D.

Further, the bending portion 22 may be provided on the distal portion of the optical waveguide portion 21 as shown in FIG. 4, or the large-diameter portion 23 may be provided on the proximal portion of the optical waveguide portion 21 as shown in FIGS. 5A to 5C. With such a configuration, the distal and proximal movement of the tubular member 30 with respect to the optical waveguide portion 21 is restricted, and thus, the endoscope 20 and the tubular member 30 are fixed. Although not shown, the optical waveguide portion 21 may be provided with both the bending portion 22 and the large-diameter portion 23 in order to firmly fix the endoscope 20 and the tubular member 30.

Besides, the endoscope 20 and the tubular member 30 can also be fixed by another method such as welding, fusing, fitting, engagement, and bonding. Further, the tubular member 30 may have a protrusion or a recess, the endoscope 20 may have a protrusion or a recess, and the protrusion of the tubular member 30 may be engaged with the recess of the endoscope 20, or the recess of the tubular member 30 may be engaged with the protrusion of the endoscope 20.

It is preferable that, at the distal end 30A of the tubular member 30, a gap between the tubular member 30 and the optical waveguide portion 21 is 0.5 times or more and 2 times or less a gap between the tubular member 30 and the lacrimal duct tube 10 in the radial direction of the tubular member 30.

In order to increase the slipperiness of the endoscope 20 with respect to the lacrimal duct tube 10, the tubular member 30 is preferably made of a synthetic resin, and is more preferably made of at least one of a polyimide resin, a fluorine resin, a polyethylene resin, and a polyamide resin. When a polyimide resin or the like is used, the strength of the optical waveguide portion 21 of the endoscope 20 enclosed by the tubular member 30 can be increased. Further, the tubular member 30 may have a slit and may be made of metal. For example, as the tubular member 30, a metal member having a slit parallel to the axial direction of the optical waveguide portion 21 can be used. When the tubular member 30 is a metal member, it has high strength, which is useful for protecting the optical waveguide portion 21. When having a slit, the tubular member 30 is flexible, whereby the tubular member 30 can be easily attached to and detached from the optical waveguide portion 21. While a slit parallel to the axial direction of the optical waveguide portion 21 as described above can be used as the slit, a spiral slit starting from the proximal end part of the tubular member 30 and terminating at the distal end part thereof can be preferably used.

The thickness of the tubular member 30 can be set according to the inner diameter of the lacrimal duct tube 10, and it is preferably 50 μm or more, more preferably 60 μm or more, and still more preferably 70 μm or more. Further, it is preferably 150 μm or less, more preferably 120 μm or less, and still more preferably 100 μm or less. With this configuration, the endoscope 20 can be inserted into the lumen of the lacrimal duct tube 10 with the tubular member 30 being attached to the optical waveguide portion 21 of the endoscope 20.

In order to increase the slipperiness of the tubular member 30 with respect to the lacrimal duct tube 10, a lubricant may be applied to the outer surface of the tubular member 30. However, from the viewpoint of preventing the lubricant from being separated due to contact between the lacrimal duct tube 10 and the tubular member 30 or between the lacrimal duct tube 10 and the endoscope 20, or preventing a hydrophilic lubricant from being eluted and becoming a foreign substance in the lacrimal duct 50, it is preferable that a lubricant is not applied on the outer surface of the tubular member 30 or the outer surface of the optical waveguide portion 21 of the endoscope 20. For the same reason, a lubricant may not be applied onto the inner surface of the lacrimal duct tube 10.

As shown in FIGS. 6 and 7, it is preferable that the openings 11 are provided at the first end part and the second end part of the lacrimal duct tube 10, and the outer diameter of the tubular member 30 at the distal end 30A is larger than the diameter of the opening 11. In that case, it is preferable that the distal end 20A of the endoscope 20 is located distal to the opening 11. With this configuration, the opening 11 of the lacrimal duct tube 10 is caught by the distal end 30A of the tubular member 30, so that the lacrimal duct tube 10 can be delivered to a desired position while puncturing and penetrating the obstructed site within the lacrimal duct 50 by the distal end part of the endoscope 20.

The inner diameter of the lacrimal duct tube 10 is preferably constant in order to prevent an excessive contact of the endoscope 20 with the inner surface of the lacrimal duct tube 10 when the endoscope 20 is removed from the lacrimal duct tube 10. For the same reason, it is preferable that the inner surface of the lacrimal duct tube 10 is flat, and it is also preferable that no step is provided.

It is preferable that the opening 11 of the lacrimal duct tube 10 in the first section 10A or the opening 11 in the second section 10B is located distal to the proximal end 30B of the tubular member 30. With this configuration, a part of the tubular member 30 on the proximal portion can be exposed. As a result, in the procedure, a method in which, after the tubular member 30 is disposed within the lacrimal duct tube 10, the optical waveguide portion 21 is inserted into the lumen of the tubular member 30 can be selected, in addition to the method in which, after the tubular member 30 is attached to the optical waveguide portion 21 of the endoscope 20, the optical waveguide portion 21 is inserted into the lumen of the lacrimal duct tube 10. Note that FIGS. 3A to 3D show examples in which the proximal end surface 10AP of the lacrimal duct tube 10 in the first section 10A is located distal to the proximal end 30B of the tubular member 30.

As shown in FIG. 4, the bending portion 22 is preferably covered with the tubular member 30. This is because friction between the optical waveguide portion 21 and the inner wall of the lacrimal duct tube 10 tends to increase particularly in the vicinity of the bending portion 22.

It is preferable that, in a case where the bending portion is provided at the distal end part of the optical waveguide portion 21 of the endoscope 20, the distal end 30A of the tubular member 30 is located distal to the bending portion 22. The endoscope 20 is likely to come into contact with the inner wall of the lacrimal duct tube 10 at the bending portion. However, due to the bending portion 22 being covered with the tubular member 30, the endoscope 20 can be easily inserted and removed.

Another embodiment of the tubular member 30 will be described with reference to FIG. 10. FIG. 10 is a side view showing a modification of the tubular member 30. As shown in FIG. 10, a groove 32 may be provided in the outer surface of the tubular member 30. This configuration reduces a contact area between the inner wall of the lacrimal duct tube 10 and the tubular member 30, thereby facilitating removal of the endoscope 20 from the lacrimal duct tube 10.

The groove 32 preferably extends along the longitudinal axis direction of the tubular member 30. When the groove 32 extends as described above, friction between the inner wall of the lacrimal duct tube 10 and the outer surface of the tubular member 30 can be reduced.

The groove 32 can be curved or linearly formed, and it is particularly preferable that the groove 32 is spirally formed. When the groove 32 is formed as described above, friction between the inner wall of the lacrimal duct tube 10 and the outer surface of the tubular member 30 can be reduced.

The tubular member 30 may be provided with a plurality of grooves 32. When the plurality of grooves 32 is provided, friction between the inner wall of the lacrimal duct tube 10 and the outer surface of the tubular member 30 can be further reduced.

FIGS. 11A and 11B show another example of the lacrimal duct tube 10 according to the embodiment of the present invention (FIG. 11A shows a plan view and Fig.11B shows a side view). In FIGS. 11A and 11B, the lacrimal duct tube 10 has a first tubular portion 55 located at the first side, a second tubular portion 55 located at the second side, and a central portion 56 located between the first tubular portion 55 and the second tubular portion 55, and at least one of the first tubular portion 55 and the second tubular portion 55 has a scale 57 for specifying the position of the lacrimal duct tube 10 in the longitudinal direction. Preferably, each of the first tubular portion 55 and the second tubular portion 55 has the scale 57 for specifying the position of the lacrimal duct tube 10 in the longitudinal direction. The scale 57 may be colored on the outer surface of the tubular portion 55, or may be formed by mixing a dye such as a pigment with the resin constituting the tubular portion 55. When the lacrimal duct tube 10 configured as described above is used, the following effect can be obtained. Specifically, when the first end of the lacrimal duct tube 10 is inserted into the nasolacrimal canal 54, and then the second side of the lacrimal duct tube 10 is inserted into the nasolacrimal canal 54, the operator can easily insert the second tubular portion 55 of the lacrimal duct tube 10 along the first tubular portion 55 which has been inserted first, while checking, with the endoscope 20, the scale 57 of the lacrimal duct tube 10 which has been inserted first. As described above, in the configuration in which the distal end 21A of the optical waveguide portion 21 is located distal to the distal end 30A of the tubular member 30, the viewing angle of the optical waveguide portion 21 is sufficiently ensured, whereby it is easier to see the scale 57. Therefore, the tubular portion 55 to be inserted later can be easily moved forward.

This application claims the benefit of the priority date of Japanese patent application No. 2018-157488 filed on Aug. 24, 2018. All of the contents of the Japanese patent application No. 2018-157488 filed on Aug. 24, 2018 are incorporated by reference herein.

### REFERENCE SIGNS LIST

1: Lacrimal intubation system
10: Lacrimal duct tube
10A: First section
10B: Second section
10C: Central section
10D: First end of the lacrimal duct tube
10AP: Proximal end surface of the first section
10BP: Proximal end surface of the second section
11: Opening
12: Insertion port
20: Endoscope
20A: Distal end of the endoscope
21: Optical waveguide portion
21A: Distal end of the optical waveguide portion
21B: Proximal end of the optical waveguide portion
22: Bending portion
23: Large-diameter portion
24: Objective lens
25: Illumination lens
26: Liquid injection channel
27: Outer tube
28: Operating portion
30: Tubular member
30A: Distal end of the tubular member
30B: Proximal end of the tubular member
31: Tubular member
32: Groove
40: Fixing member
50: Lacrimal duct
51: Lacrimal punctum
52: Lacrimal canaliculus
53: Lacrimal sac
54: Nasolacrimal canal
55: Tubular portion
56: Central portion
57: Scale

1. A lacrimal intubation system comprising:
a lacrimal duct tube having a first side and a second side in a longitudinal direction, each of the first side and the second side being insertable into a lacrimal duct and having a proximal end and a distal end;
an endoscope having an optical waveguide portion extending in a longitudinal direction, the optical waveguide portion being configured to be inserted into a lumen of the lacrimal duct tube; and
a tubular member enclosing the optical waveguide portion.

2. The lacrimal intubation system according to claim 1, wherein the tubular member is disposed in the lumen of the lacrimal duct tube.

3. The lacrimal intubation system according to claim 1, wherein
the optical waveguide portion has a distal end and a proximal end,
the tubular member has a distal end and a proximal end, and
the optical waveguide portion and the tubular member are configured such that the distal end of the optical waveguide portion is located at a more distal side than the distal end of the tubular member.

4. The lacrimal intubation system according to claim 1, wherein
the lacrimal duct tube and the endoscope are configured such that the endoscope is slidably movable in the lumen of the lacrimal duct tube in the longitudinal direction, and
the tubular member and the endoscope are configured such that the endoscope is not slidably movable in a lumen of the tubular member in the longitudinal direction.

5. The lacrimal intubation system according to claim 1, wherein
the tubular member is disposed in the lumen of the lacrimal duct tube,
the tubular member has a distal end, and at the distal end, a gap between the tubular member and the optical waveguide portion is 0.5 times or more and 2 times or less a gap between the tubular member and the lacrimal duct tube in a radial direction of the tubular member.

6. The lacrimal intubation system according to claim 1, wherein
the lacrimal duct tube and the optical waveguide portion are configured such that a distal end of the optical waveguide portion is located at a more distal side than the distal end of at least one of the first side and the second side of the lacrimal duct tube in the longitudinal direction of the lacrimal duct tube.

7. The lacrimal intubation system according to claim 1, wherein
the tubular member has a proximal end aligned with a proximal end of the optical waveguide portion.

8. The lacrimal intubation system according to claim 1, wherein
the tubular member has a distal end and a proximal end, and
the tubular member and the optical waveguide portion are configured such that the proximal end of the tubular member is located between a proximal end of the optical waveguide portion and the distal end of the optical waveguide portion.

9. The lacrimal intubation system according to claim 1, wherein
at least a part of the optical waveguide portion is made of metal.

10. The lacrimal intubation system according to claim 1, wherein
the lacrimal duct tube has a first tubular portion located at the first side, a second tubular portion located at the second side, and a central portion located between the first tubular portion and the second tubular portion, and
each of the first tubular portion and the second tubular portion has a scale for specifying a position of the lacrimal duct tube in the longitudinal direction.

11. The lacrimal intubation system according to claim 1, wherein
the tubular member is made of at least one of a polyimide resin, a fluorine resin, a polyethylene resin, and a polyamide resin.

12. The lacrimal intubation system according to claim 1, wherein
the tubular member has a slit, and is made of metal.

13. The lacrimal intubation system according to claim 1, wherein
the lacrimal duct tube has a first tubular portion located at the first side, a second tubular portion located at the second side, and a central portion located between the first tubular portion and the second tubular portion, and
the lacrimal duct tube and the endoscope are configured such that the optical waveguide portion is slidably movable in a lumen of at least one of the first tubular portion and the second tubular portion of the lacrimal duct tube.
